(19) United States
(12) Patent Application Publication
(10) Pub. No.: US 2024/0252217 A1
(43) Pub. Date: Aug. 1, 2024

(54) Kit for the installation of prosthetic components and/or biomedical implants

(71) Applicant: MT Ortho S.r.l., ACI Sant'Antonio (CT) (IT)

(72) Inventors: Simone Di Bella, ACI Sant'Antonio (CT) (IT); Carmine Zoccali, Reggio Calabria (RC) (IT)

(21) Appl. No.: 18/563,908

(22) PCT Filed: May 27, 2022
(86) PCT No.: PCT/IB2022/054988

§ 371 Date: Nov. 23, 2023

(30) Foreign Application Priority Data
May 27, 2021 (IT) 102021000013841

(51) Int. Cl. A61B 17/84; A61F 2/30

(52) U.S. Cl. CPC A61B 17/842; A61F 2/30942; A61F 2002/30159

(57) ABSTRACT

A kit for installation of prosthetic components and/or biomedical implants includes a prosthetic component having an intraosseous taproot, formed of one piece with the prosthesis, for the attachment of the prosthetic component to the bone of a patient. The prosthetic component and the taproot are made of a biocompatible metal material, and the taproot has at least one portion with a triangular cross-section, as viewed in a horizontal cross-sectional plane. At least one first guiding component for a guide wire, for guiding the insertion of a guide wire into the bone, and at least one second guiding component for an impactor having a triangular section are also included.

### FIELD OF THE INVENTION

The present invention relates to a kit for installation of prosthetic components and/or biomedical implants comprising a prosthetic component having an intraosseous taproot formed of one piece with the prosthesis, for attachment of said prosthetic component to the bone of a patient.

The intraosseous biomedical device for attachment of the prosthetic component, also known in the art as taproot, is made of a biocompatible metal material such as titanium and/or alloys thereof, suitable for use in biomedical implants, namely for attachment of prosthetic components or prosthetic parts, such as, without limitation, orthopedic prostheses, to the bone.

By way of example and without limitation, the kit for installation of prosthetic components and/or biomedical implants of the present invention may be used in the installation of acetabular prosthetic implants, as well as in implantation of prosthetic knees, elbows, shoulders, pelvis, and so on.

### Background Art

A number of known biomedical applications use taproots, that are inserted into the bone to firmly fix a prosthetic component to the bone itself.

Examples of such applications are found in acetabular prostheses (FIG. 1) as well as knee prostheses (FIG. 2). In all currently known applications, taproots have a circular cross-section, as this shape allows the surgeon to make intraoperative adaptations by rotating the component, for example the cotyl or acetabulum or the tibial tray of a prosthetic knee, about the longitudinal axis of the taproot even when the letter is already inserted in the bone.

Examples of taproots having geometries other than the circular cross-section geometry, include the nails as used for bone fusion, such as for fixation of the sacroiliac joint, or the Thornton Nail, i.e. a three-flange fixation nail that was used in the '70s of the last century for treatment of femoral neck fractures.

An example of a taproot having a triangular geometry is provided in U.S. Pat. No. 9,339,394 B2, which illustrates a prosthetic vertebral facet suitable for replacement of cartilage and a bone portion of the natural vertebral joint facet. Among alternative embodiments of the fixation system, a transpedicular screw having a triangular cross-section is shown. Therefore, the taproot of this document is not rigidly joined with the prosthetic element, whereby the patent does not imply the problem of ensuring proper orientation of the prosthetic element in space in response to the orientation of the taproot, and does not show or suggests the use of guide components for proper positioning of the prosthetic element,.

No example of systems or kits are currently available which use taproots with a section other than the cylindrical section in intraosseous fixation systems, for reconstruction/replacement of bone parts, because the circular section can roughly fit any anatomy, and can thus afford series production in different sizes, with the surgeon having the responsibility for optimized positioning of the taproot in the bone of the patient, and thus for orientation of the prosthetic element associated with the taproot.

Nevertheless, these known arrangements still suffer from certain drawbacks.

These drawbacks mainly include the inability of conventional circular-section taproots to resist torsional loads on the prosthesis. A stem having a cylindrical section opposes low resistance to torsional strain, only provided by the friction between the stem and the inner surfaces of the bone contacted by said stem.

Furthermore, prior art circular section-taproots leave responsibility to the surgeon, who has no reference and shall use fluoroscopy, which in fact exposes the surgeon him/herself and the patient to X-rays

Therefore, the present patent application has the purpose to obviate the residual drawbacks of the prior art, as more clearly discuss4ed hereinafter.

### SUMMARY OF INVENTION

The main purpose of the present invention is to provide a kit for installation of prosthetic components and/or biomedical implants, wherein said prosthetic component comprises a taproot for intraosseous fixation of the prosthetic component in reconstruction and/or replacement of bone parts, which can solve or at least reduce the drawbacks of prior art systems.

In the pursuance of this purpose, an object of the present invention is to provide a prosthetic component comprising a taproot for intraosseous fixation of the prosthetic component, that is able to withstanding torsional loads.

A further object of the present invention is to provide a prosthetic component comprising a taproot for intraosseous fixation of the prosthetic component, that affords unique positioning of the taproot and of the prosthetic component associated therewith relative to the anatomical site of the patient with which the prosthesis is to be associated, thereby guiding the surgeon in positioning the taproot and hence the prosthetic component, and dramatically reducing the risk of wrong positioning and optimizing the surgery, thereby affording bone reconstruction even in highly complex situations.

The above purposes and these and other objects of the present invention are fulfilled by a kit for installation of prosthetic components and/or biomedical systems, wherein said prosthetic component comprises a taproot for intraosseous fixation of the prosthetic component in reconstruction and/or replacement of bone parts, thereby affording superior stability of the implant and excellent load-bearing capacity even after a short time from reconstruction.

The above purpose and these and other objects of the present invention are fulfilled by a kit for installation of prosthetic components comprising a taproot for intraosseous fixation as defined in claim 1.

Further characteristics of the kit and the prosthetic component comprising a taproot for intraosseous fixation according to the present invention will form the subject of the dependent claims.

### LIST OF FIGURES

The characteristics and advantages of the kit for installation of prosthetic components and/or biomedical implants comprising a taproot made of a biocompatible metal material for the intraosseous fixation of said prosthetic component according to the present invention and of the prosthetic component comprising said taproot according to the present invention will be more readily apparent upon reading of the following detailed description, given by way of example and without limitation, with reference to the accompanying schematic drawings, in which:

FIGS. 1 and 2 show examples of prior art circular-section taproots;

FIG. 3 shows a front view of the taproot which equips the prosthetic components of the present invention;

FIG. 4 shows a front view of the taproot of FIG. 3, which shows the central body only, without the trabeculated portion;

FIG. 5 is a longitudinal sectional view as taken along a vertical plane A-A as shown in FIG. 4;

FIG. 6 is a top plan view of the taproot of FIG. 3;

FIGS. 7 and 8 show perspective views of the taproot of the present invention which equips an acetabular prosthetic component;

FIG. 9A shows a front view of an example of a first guide wire-guiding element according to an embodiment suitable for installation of an acetabular prosthetic component;

FIG. 9B is a perspective view of the guide wire-guiding element of FIG. 9B;

FIG. 9C shows a step of Computer Tomography imaging of the surgery zone for defining the design of the guide wire-guiding element;

FIG. 10 shows the guide wire-guiding element of FIGS. 9A and 9B of the present invention, properly seated and oriented, and associated with an acetabular seat of the pelvis, with the guide wire inserted;

FIG. 11A shows a front view of an example of a second guide element for a triangular impactor;

FIG. 11B is a perspective view of the triangular impactor-guiding element of FIG. 9B;

FIG. 11C shows a step of Computer Tomography imaging of the surgery zone for defining the design of the triangular impactor-guiding element;

FIG. 12 shows the triangular impactor-guiding element of FIGS. 11A and 11B of the present invention, properly seated and oriented, and associated with an acetabular seat of the pelvis;

FIGS. 13 and 14 show the taproot of the present invention associated with an inserted acetabular prosthesis associated with the patient's pelvis, with the addition of possible additional fixation screws;

FIGS. 15 to 26 show the steps of preparing the surgery site for reconstruction of the left femur-acetabulum joint by means of a fixation kit of the present invention comprising guide components for preparation of the implantation site and a prosthetic element with of a pair of triangular-section taproots according to the present invention.

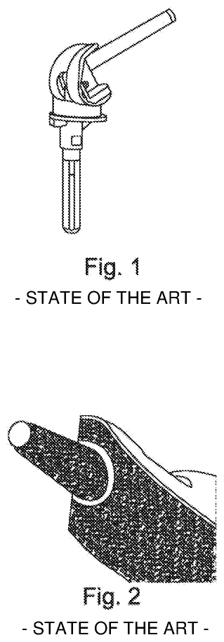

### DETAILED DESCRIPTION OF THE INVENTION

The present invention relates to a kit 100 for installation of prosthetic cotyloid components 40, 50 in which said prosthetic component 40, 50 comprises one or more taproots 10 depending on the extent of the osteotomy for intraosseous fixation of the prosthetic component itself in reconstruction and/or replacement of bone parts.

The prosthetic component 40, 50 that is part of the kit 100 of the present invention, comprises a taproot 10 for intraosseous fixation, that is formed in one piece with said prosthetic component 40, 50.

The prosthetic component 40, 50 and the taproot 10 are monolithic constructions made of a biocompatible metal material, more particularly titanium- and/or titanium alloy-based metal materials.

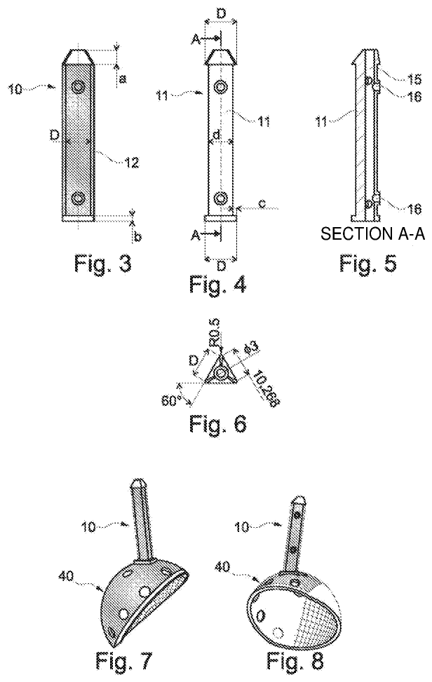

Advantageously, the taproot 10 which equips the prosthetic component 40, 50 of the kit 100 of the present invention, is manufactured using the 3D EBM (Electron Beam melting) additive manufacturing technology and has a structure comprising a solid central body 11 with a triangular cross-section, as viewed in a substantially horizontal plane, and a trabeculated portion 12, as shown for example in FIG. 3, rigidly joined to said central body 11.

Advantageously, the taproot 10 of the present invention has at least one portion with a substantially triangular cross section as taken along a horizontal plane. As shown in the top view of FIG. 6, and in the perspective views of FIGS. 7 and 8, according to a preferred embodiment of the present invention, the entire length of the taproot 10 of said prosthetic component 40, 50, including the end sections 11a, 11b, has a triangular cross-section.

Advantageously, the taproot 10 of said prosthetic component 40, 50 of the present invention further comprises, inside said central body 11, a longitudinal through hole 15 for receiving a guide wire during implantation.

Advantageously, the taproot 10 may further comprise one or more additional discharge holes 16 in communication with said axial hole 15, for putting said axial hole 15 in communication with the outside, in order to create an exit path for any bone cement, platelet concentrate or stem cells injected through the central hole 15 once the latter has been used as a guide for the guide wires, to stabilize the system if the surgeon is not satisfied or confident with the purely mechanical holding strength of the taproot, for example due to the consistency of the bone tissue.

The aforementioned discharge holes 16 may advantageously have an inside diameter of 2.5 mm and an outside diameter of 4.5 mm.

Still referring to the accompanying figures, the taproot 10 of the present invention will comprise, at its front end 11a, an end element 13 having a frustoconical profile along a vertical plane as shown in FIGS. 3 to 5, and having a height a that is preferably about 5 mm. This frustoconical front end element 13 is configured to facilitate insertion of the taproot into the medullary channel of the patient's bone.

On the other hand, at the rear end 11b of said taproot 10, designed for connection to the prosthetic element to be associated with the bone, a base element 14 may be provided, having a width D along a vertical longitudinal plane and a height b of preferably about 2 mm.

According to a preferred embodiment of the present invention, the taproot 10 is formed in one piece with the prosthetic component 40, 50, advantageously using the EBM technology.

Advantageously, as shown in the accompanying figures, the aforementioned front end 11a of said taproot has a frustoconical shape 13 with a proximal larger base 13a and a distal smaller base 13b.

Said frustoconical end element 13 is rigidly joined to or, advantageously, formed in one piece by EBM with said central solid core 11, and the width of its larger base 13a is equal to the overall width D of the taproot.

Advantageously, the width D of the larger base 13a is greater than the width d of the cylindrical central core 11, still considered on a vertical plane like that of the front views of FIGS. 3 and 4, with the difference c=D−d indicated in FIG. 4 and preferably of more than 1 mm, so that the trabeculated portion 12 formed in one piece by EBM with said central cylindrical body 11 will not exceed the width D of said larger base 13a.

Thus, the trabeculated portion 12 extends around said central body 11 having a triangular cross-section while remaining within the shape defined by the two end elements 13, 14 of the taproot, as shown for example in FIG. 3.

By this arrangement, intraosseous insertion is not likely to cause damage to or rupture of the trabeculae.

FIG. 6, which shows a top view of the taproot 10 of the present invention, illustrates the triangular cross-sectional configuration of the frustoconical end element 13.

The axial length of the taproot 10 of the present invention may vary according to the anatomical site and/or the specific needs of the patient, advantageously in a range of 20 mm to 100 mm.

The above described configuration of the taproot 10 of the present invention, which comprises a solid central body 11 and a trabeculated portion 12, also affords press- or interference-fit insertion of the taproot into the bone during surgery, which provides load resistance, as well as osteointegration over time and stimulation of bone due to the trabeculated portion.

One study (Int. J. Mol. Skiing. 2021, 22, 2379; “Superior Osteo-Inductive and Osteo-Conductive Properties of Trabecular Titanium vs. PEEK Scaffolds on Human Mesenchymal Stem Cells: A Proof of Concept for the Use of Fusion Cages”) conducted on titanium scaffolds manufactured by the applicant hereof has confirmed the superior osteoinductive and osteoconductive properties of the Ti6Al4V ELI trabeculated titanium structure.

As mentioned above, the prosthetic component 40, 50 comprising the taproot 10 of the present invention is advantageously formed by manufacturing techniques that include localized melting of (metal or polymer) powders using high-energy electron beams.

These techniques, known as EBM (Electron Beam Melting), are leading-edge manufacturing technologies which can form objects with very complex geometries and different surface roughnesses from a computer design of the finished product, which is processed by computerized machines that guide the electron beam in its action.

Electron beam melting is a relatively new rapid prototyping technique for the production of implant structures, and affords complex three-dimensional geometries.

Using this technique, the Applicant hereof has developed the kit 100 of the present invention, in which the prosthetic component 40, 50 comprises the taproot 10 with the part with the trabeculated structure 12 having pore sizes between trabeculae in the order of one hundred microns.

More particularly, the preferably regular trabeculated structure 12 will have a pore diameter ranging from 400 to 800 microns, more preferably the pore diameter will be about 600 microns, preferably 640 microns.

Namely, the titanium or titanium alloy trabeculated structure 12, whose elastic modulus is very proximate to that of the natural trabecular bone, restores physiological load transfer, thereby preventing bone damage and even promoting bone regrowth.

It should be noted that the EBM technology allows the trabeculated portion 12 to be formed in one piece with the central body 11.

The Applicant has also found that the particular configuration of the taproot 10 of the present invention, namely the provision of a central body 11 having a solid structure connected at its ends to the end portions 13, 14 as described, and of the large trabeculated portion 12 extending around said central body 11, allows the implant to have an optimal mechanical load-bearing behavior in vivo, thereby immediately achieving primary stability of the plant, due to both the structure of the taproot 10 and to its triangular cross-section which provides a superior resistance to torsional loads.

Also, the outermost trabeculated portion 12 both ensures osteointegration of the system in the weeks following surgery, and further improves primary stability and “grip”, i.e. the friction that opposes movements and in particular taproot withdrawal, since the first postoperative stages.

The taproot 10 of the present invention comprises, as mentioned above, an axial through hole 15, allowing guided device implantation using a guide wire and, if needed, allowing the surgeon to inject a certain amount of biocement into the taproot to further improve stability of an otherwise simple press-fit.

Also, the triangular cross-section provides significant biomechanical benefits as compared with known taproot types, such as the ability to resist torsional loads, and to facilitate a surgeon, in properly positioning the implant with appropriate instruments relative to the bone, by affording unique positioning, and thus reducing the importance of manual orientation which, in known systems, is defined by the surgeon during surgery, resulting in the above discussed problems.

This specific advantage is achieved by a kit 100 that comprises the taproot 10 of the present invention and additional guide elements forming the instruments that guide the surgeon during prosthesis implantation.

The aforementioned kit 100 is also encompassed by the present invention and comprises, in addition to said taproot 10, a first guide wire-guiding component 20, for guiding insertion of a guide wire, and a second guiding component 30 for an impactor having a triangular cross-section.

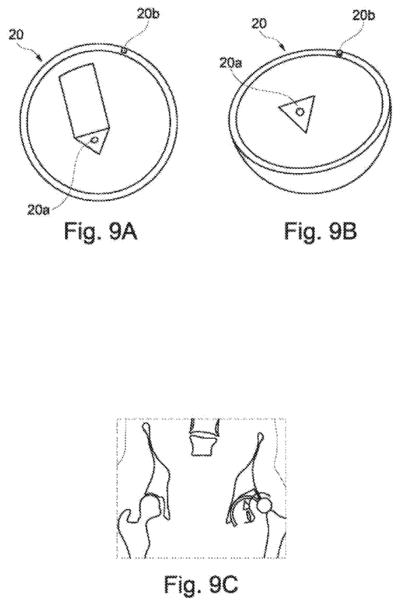

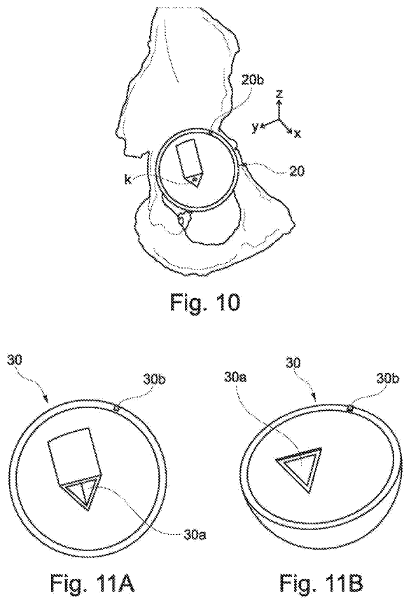

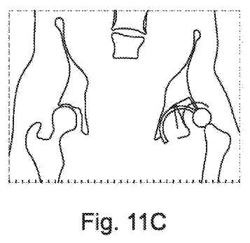

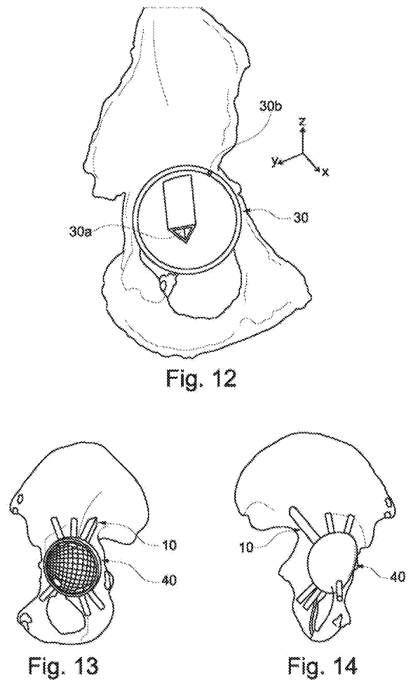

Reference is particularly made to FIGS. 9A, 9B and 10 which show the first guide wire-guiding component 20, and FIGS. 11A, 11B and 12, which show the second guiding component 30 for a triangular impactor adapted to form the seat for the taproot 10 in the bone. As shown in the figures, the first guide wire-guiding component 20 advantageously comprises a seat 20a for receiving the guide wire k and a peripheral marker 20b, whereas the second triangular impactor-guiding component 30 comprises a first seat 30a for receiving the guide wires that have been already positioned in the bone and the impactor, and also a peripheral marker 30b. These references that guide the surgeon in component orientation ensure proper positioning of the guide elements and hence proper orientation of the taproot seat that is formed in the bone for receiving the triangular taproot 10.

Each of said first and second guiding components 20, 30 is advantageously designed according to the surgical procedure to be performed.

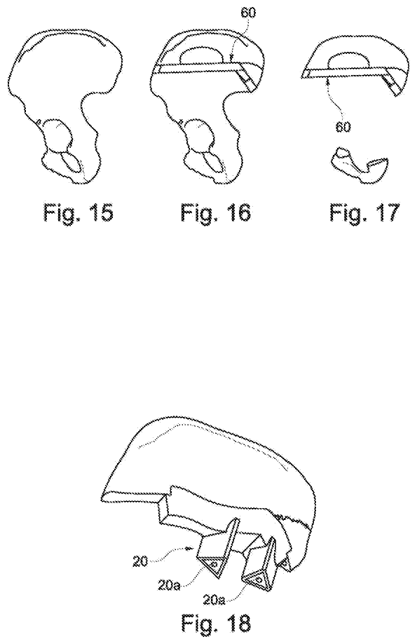

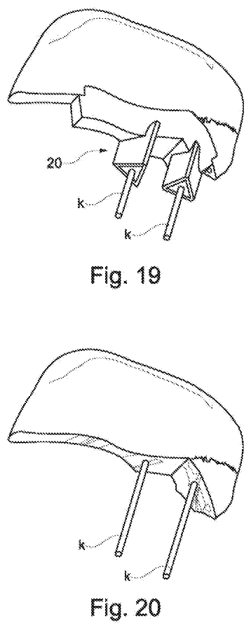

Thus, for example, FIGS. 18-20 show a different embodiment of said first guide wire-guiding component 20, which is specifically configured for a procedure including, as shown in FIGS. 15-17, osteotomy of the pelvis bone using an anatomical template 60, such osteotomy possibly covering a large portion of the bone, as may be the unfortunate case of bone cancer.

Said first guide wire-guiding component 20 is configured to perfectly fit the shape of the edge of the bone on which it is designed to rest.

FIG. 21 shows a different embodiment of said second guiding component 30 for an impactor having a triangular cross-section, specially designed for the procedure, so as to perfectly fit the shape of the edge of the bone on which it is designed to rest.

In order to manufacture custom-made components that can perfectly fit the bone surfaces to be contacted, to thereby ensure unique, and hence failsafe positioning, said guiding components 20, 30 are also advantageously manufactured with techniques providing localized melting of (metal or polymer) powders using high-energy electron beams, known as EBM.

Thus, said first and second guiding components 20, 30 may be advantageously custom-made to tailor the patient and the type of procedure to be performed, based on a CT scan for imaging the anatomical site in which the prosthesis is to be implanted and planning of the procedure, the guide elements being also accordingly custom-made, for guiding proper positioning of the prosthesis.

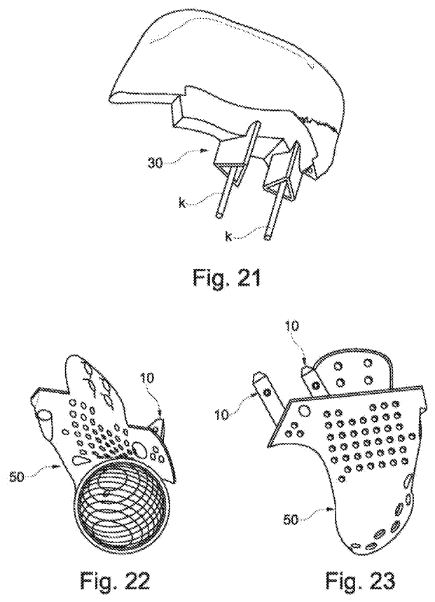

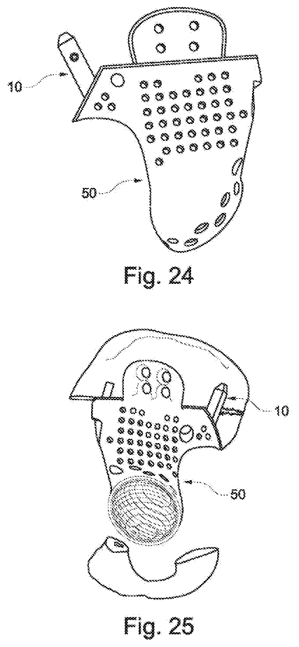

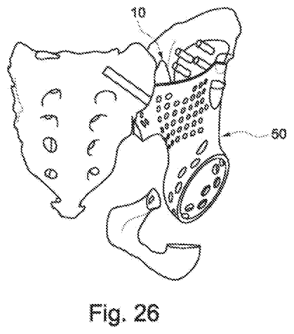

FIGS. 22 to 26 illustrate by way of example a prosthesis 50 associated with a taproot 10 of the present invention, specifically designed for reconstruction of the femur-acetabular joint as needed to restore the functionality of the joint which had to be completely eliminated due to the selected type of procedure.

While an embodiment of custom-made manufacture of the guiding components 20, 30 will be described below, affording design and performance of even highly complex bone and/or joint reconstruction procedures, such as the one as shown in the accompanying figures, it shall be understood that said taproot 10 and said guiding components 20, 30 may also be mass-produced, advantageously in different sizes, thereby affording the advantage of ensuring superior attachment of the prosthesis to the bone by means of the taproot.

A kit comprising a taproot 10 and custom-made guiding components 20, 30 particularly provides additional benefits as compared with mass-produced components, such benefits being highly appreciated and/or affording procedures that would otherwise be unfeasible with standard mass-produced components.

These benefits, as discussed above, may be summarized as follows:
very high shape precision to ensure the highest precision in the contact surfaces between the prosthesis and the bone;
no need to change or adapt positioning of the components (taproot and guide components) during the procedure, which allows the surgeon to be assured of proper prosthesis positioning even under very difficult procedure conditions;
excellent biointegrative and osteoinductive properties, due to the trabeculated structure of the taproot and high mechanical resistance also to torsional loads due to the solid central portion and the triangular section;
radiopacity and low infection risk rate.

In order to produce custom-made components, the captured images are processed by three-dimensional image processing software and the design of the custom-made guiding components 20, 30 is made.

Therefore, the process for custom-made production of the guide wire-guiding component 20 and the impactor-guiding component 30 may advantageously comprise the steps of:
capturing CT scan images of the patient;
processing the CT images, followed by segmentation and production of a 3D model of the bone tissue of the patient;
designing custom-made guiding components 20, 30.

The above described method of manufacturing the guiding components 20, 30 is focused on restoration of the anatomy and functionality of the anatomical site to be treated, and also affords proper attachment of the taproot 10 and the prosthetic element associated therewith to the bone tissue, thereby ensuring prosthesis stability over time.

The design of specific instruments, namely the guiding components 20, 30, can guide the surgeon in replicating as faithfully as possible the in-situ positioning of the biomedical device as defined in the pre-operative planning stage, in the operating room.

Such guiding components 20, 30, as well as the device including the taproot 10 may be designed in a patient-specific manner, as mentioned above, to have mating surfaces perfectly adhering to the host anatomical site, thereby affording the surgeon fixed, unique positioning features, preventing any risk of positioning errors and facilitating the procedure, as the surgeon is not required to change or adapt component positioning during surgery.

The method of using the guiding components 20, 30 according to the invention comprises at least the steps of:
using said first guiding component 20 to allow insertion of a guide wire K in the direction established when designing the surgical procedure;
then, while holding the guide wire K in place, positioning the second guide component 30, which acts as a guide for proper orientation of an impactor having a triangular section, thereby forming the seat for the taproot 10.

Successive use of the two guiding components 20, 30 of the invention affords unique definition of the direction and orientation of the taproot 10 and accordingly of the prosthetic component 50, which is rigidly joined thereto.

Finally, the taproot 10 is implanted in a guided manner, due to the presence of the axial through hole 15 which allows insertion of the guide wire K, and is then inserted by interference press-fit into the seat formed in the bone using an impactor guided by said second guiding component 30.

The kit 100 comprising the taproot 10 configured, as described above, by the two guiding components 20, 30 can replicate during surgery what has been defined in the pre-operative design stage, thus guiding the surgeon in the implantation of the taproot and of the prosthetic component associated therewith.

In case of custom-made prostheses and components, all the components of the kit are designed based on the anatomy of the patient, and in particular based on the morphology of the specific implantation area.

The positioning of the two custom-made guiding components 20, 30 and then of the taproot 10 will be unique as the contact surfaces of the guiding components relative to the bone surface will be unique and perfectly complementary.

The characteristics and advantages of the taproot 10 and the kit 100 comprising such taproot of the present invention will be apparent from the above description.

It was particularly shown that the kit 100 of the present invention, and particularly the guiding elements 20, 30 mass-produced in different sizes or custom-made according to the specific anatomical conformation of the patient, can guide the surgeon in the installation of the triangular taproot 10, and hence of the associated prosthetic component, with a dramatic reduction or elimination of positioning error risks.

Thus, the dedicated instruments, comprising said guiding elements 20, 30 allow the use of a triangular taproot 10 as described above which, as already mentioned, will provide considerable benefits, including higher resistance to torsional loads and greater precision in positioning the prosthesis to perfectly match with what was established during pre-operative planning.

It shall be understood that the intraosseous triangular taproot thus conceived is susceptible of modifications and/or variants, all of which are encompassed by the invention, whose scope is defined by the accompanying claims.

In particular, the materials described, as well as the dimensions, may vary as needed.

The invention claimed is:

1. A kit (100) for installing prosthetic components (40, 50) and/or biomedical implants, said kit (100) comprising:
a prosthetic component (40, 50) comprising, made as one piece, an intraosseous taproot (10) for fixation of said prosthetic component (40, 50) to a bone of a patient, said prosthetic component (40, 50) and said intraosseous taproot (10) being made of a biocompatible metal material,
wherein said intraosseous taproot (10) of said prosthetic component (40, 50) comprises a solid central body (11) comprising an axial through-hole (15) and, externally to said central body (11), a trabecular portion (12) solidly connected to, or made of a one piece with, said central body (11), and
wherein the kit has, viewed in a horizontal cross-section, at least one seat having a triangular cross-section; and
a first guide component (20) for guide wire to guide an insertion of a guide wire (K) into the bone, and a second guide component (30) for an impactor having a triangular cross-section.

2. The kit (100) as claimed in claim 1, wherein each of said first and said second guide components (20, 30) is made in a patient-tailored manner, by processing images from a Computed Tomography scan of an anatomy of the patient.

3. The kit (100) as claimed in claim 1, wherein each of said first and said second guide components (20, 30) is designed to uniquely mate an anatomical site of prosthesis implantation, thereby ensuring a proper positioning of guide elements, and hence of the guide wire and the impactor having the triangular cross-section, thereby ensuring a proper orientation of the seat having the triangular cross-section inside the bone and a proper positioning of the intraosseous taproot (10).

4. The kit (100) as claimed in claim 1, wherein said prosthetic component (40, 50) and said intraosseous taproot (10) are made as one piece.

5. The kit (100) as claimed in claim 4, wherein said prosthetic component (40, 50) and said intraosseous taproot (10) are made as one piece using electron beam melting (EBM) technologies.

6. The kit (100) as claimed in claim 1, wherein said intraosseous taproot (10) has the triangular cross-section along its entire length, as viewed in a horizontal plane.

7. The kit (100) as claimed in claim 1, wherein said intraosseous taproot (10) has the central body (11) of triangular cross-section along at least a portion or over an entire length thereof, as viewed in a horizontal plane.

8. The kit (100) as claimed claim 1, wherein said intraosseous taproot (10) of said prosthetic component (40, 50) comprises end sections (11a, 11b) having a triangular cross-section as viewed in a horizontal plane.

9. The kit (100) as claimed in claim 1, wherein said intraosseous taproot (10) comprises, at a front end (11a) thereof, an end element (13) having a frustoconical profile as viewed in a vertical plane.

10. The kit (100) as claimed in claim 1, wherein said intraosseous taproot (10) an end element (13) with a width (D), in a vertical longitudinal plane, that is equal to an overall footprint of the intraosseous taproot (10) in the same plane.

11. The kit (100) as claimed in claim 1, wherein said intraosseous taproot (10) comprises, at a rear end (lib) of said intraosseous taproot (10) designed for connection to the prosthetic component to be associated with the bone, a base element (14) having a width (D) in a vertical longitudinal plane that is equal to an overall footprint of the intraosseous taproot (10) in the same plane.

12. The kit (100) as claimed in claim 1, wherein said intraosseous taproot (10) is made in one piece with the prosthetic element (50).

13. The kit (100) as claimed in claim 7, wherein said intraosseous taproot (10) extends around said central body (11) having the triangular cross section but remains within a shape defined by end elements (13, 14) of the intraosseous taproot.

14. The kit (100) as claimed in claim 1 wherein said trabecular portion (12) has a pore diameter that ranges from 400 to 800 microns.

15. The kit (100) as claimed in claim 1, wherein said trabecular portion (12) of said intraosseous taproot (10) is formed by EBM in one piece with the central body (11).
